(19) United States
(12) Patent Application Publication
(10) Pub. No.: US 2005/0228286 A1
(43) Pub. Date: Oct. 13, 2005

(54) Medical system having a rotatable ultrasound source and a piercing tip

(76) Inventors: Jeffrey David Messerly, Cincinnati, OH (US); Michael H. Slayton, Tempe, AZ (US); Richard P. Nuchols, Loveland, OH (US); Inder Raj S. Makin, Loveland, OH (US); Peter G. Barthe, Phoenix, AZ (US); T. Douglas Mast, Cincinnati, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/819,726

(22) Filed: Apr. 7, 2004

(51) Int. Cl. A61B 8/14

(52) U.S. Cl. 600/459

(57) ABSTRACT

An ultrasound medical system includes a handpiece and an end effector which is operatively connected to the handpiece and which is insertable into a patient. The end effector includes a shaft, a medical ultrasound transducer assembly, and a shaft head. The shaft has a longitudinal axis, has a distal end, and is rotatable about the longitudinal axis with respect to the handpiece. The transducer assembly is non-rotatably attached to the shaft and is adapted to emit medical ultrasound. The shaft head is attached to the distal end of the shaft and has a piercing tip which is insertable into patient tissue.

### FIELD OF THE INVENTION

The present invention relates generally to ultrasound, and more particularly to an ultrasound medical system having a rotatable ultrasound source and having a piercing tip insertable into patient tissue.

### BACKGROUND OF THE INVENTION

Known ultrasound medical methods include using ultrasound imaging (at low power) of patients to identify patient tissue for medical treatment and include using ultrasound (at high power) to ablate identified patient tissue by heating the tissue.

Known ultrasound medical systems and methods include deploying an end effector having an ultrasound transducer outside the body to break up kidney stones inside the body, endoscopically inserting an end effector having an ultrasound transducer in the rectum to medically destroy prostate cancer, laparoscopically inserting an end effector having an ultrasound transducer in the abdominal cavity to medically destroy a cancerous liver tumor, intravenously inserting a catheter end effector having an ultrasound transducer into a vein in the arm and moving the catheter to the heart to medically destroy diseased heart tissue, and interstitially inserting a needle end effector having an ultrasound transducer needle into the tongue to medically destroy tissue to reduce tongue volume to reduce snoring.

Rotatable ultrasonic end effectors are known wherein an ultrasound transducer is non-rotatably attached to a shaft whose distal end is circumferentially and longitudinally surrounded by a sheath having a longitudinal axis and having an acoustic window. Water between the shaft and the sheath provides acoustic coupling between the ultrasound transducer and the acoustic window. The shaft is rotatable about the longitudinal axis with respect to the sheath. The sheath is non-rotatably attached to a handpiece. In one known design, an ultrasonic imaging transducer is non-rotatably attached on one side of the shaft and an ultrasonic treatment transducer is non-rotatably attached on the opposite side of the shaft. In another known design, an ultrasonic treatment transducer of a short focal length is non-rotatably attached on one side and an ultrasonic treatment transducer of a long focal length is non-rotatably attached on the other side. A known ultrasonic end effector also includes a biopsy tool. A known non-ultrasound device is a trocar which is insertable into a patient, and which includes a protective cover to protect a sharp instrument, wherein the protective cover is self-opening exposing the sharp instrument when the protective cover is pushed against patient tissue with a force greater than a threshold force.

Known non-ultrasound medical systems include endoscopic or laparoscopic clamp end effectors, wherein the clamp end effector is articulated and is steered by the user.

Still, scientists and engineers continue to seek improved ultrasound medical systems.

### SUMMARY OF THE INVENTION

A first embodiment of the invention is an ultrasound medical system including a handpiece and an end effector. The end effector is operatively connected to the handpiece and is insertable into a patient. The end effector includes a shaft, a medical ultrasound transducer assembly, and a shaft head. The shaft has a longitudinal axis, has a distal end, and is rotatable about the longitudinal axis with respect to the handpiece. The medical ultrasound transducer assembly is non-rotatably attached to the shaft and is adapted to emit medical ultrasound. The shaft head is attached to the distal end of the shaft and has a piercing tip which is insertable into patient tissue.

Several benefits and advantages are obtained from the ultrasound medical system of the invention. The rotatable shaft having the attached medical ultrasound transducer assembly provides a rotatable ultrasound source for medically imaging and/or medically treating a greater volume of patient tissue than those systems having non-rotatable ultrasound sources. The shaft head having the piercing tip provides longitudinal immobilization (and rotational stabilization in examples where the shaft head does not rotate) of the ultrasound source (i.e., the medical ultrasound transducer assembly) to patient tissue for precise positioning for ultrasound imaging and/or treatment.

The present invention has, without limitation, application in conventional endoscopic, laparoscopic, and open surgical instrumentation as well as application in robotic-assisted surgery.

### BRIEF DESCRIPTION OF THE FIGURES

FIG. 1 is a perspective view of a first embodiment of the present invention showing an ultrasound medical treatment system which includes an end effector and a handpiece;

FIG. 2 is a schematic cross-sectional view of a first embodiment of the end effector and the handpiece of the ultrasound medical treatment system of FIG. 1;

FIG. 3 is a view, as in FIG. 2, but of a second embodiment of the end effector and the handpiece and with the protective cover and the shaft-head heating means omitted for clarity;

FIG. 4 is a view, as in FIG. 3, but of a third embodiment of the end effector and the handpiece;

FIG. 5 is a view, as in FIG. 3, but of a fourth embodiment of the end effector and the handpiece; and

FIG. 6 is a view, as in FIG. 3, but of a fifth embodiment of the end effector and the handpiece.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is understood that any one or more of the following-described embodiments, examples, etc. can be combined with any one or more of the other following-described embodiments, examples, etc.

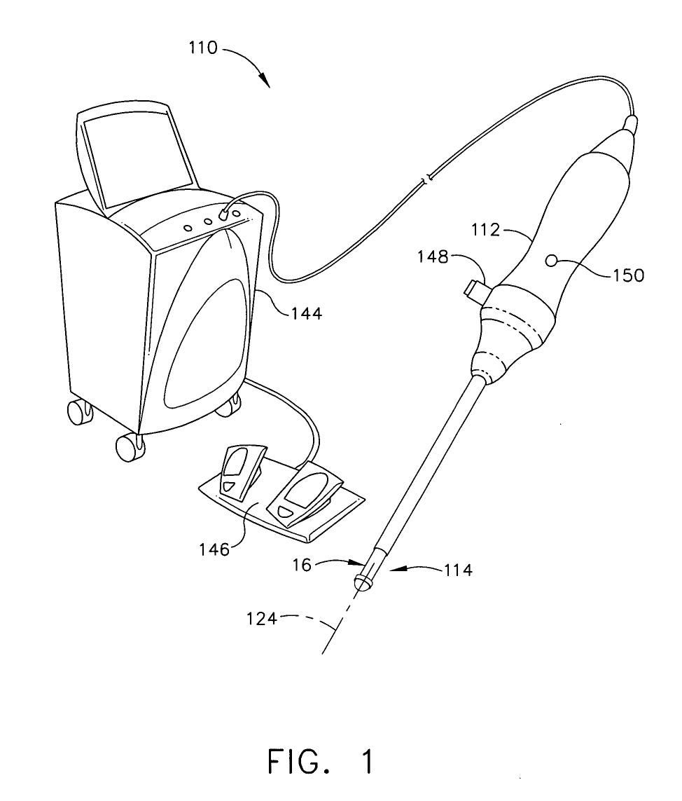

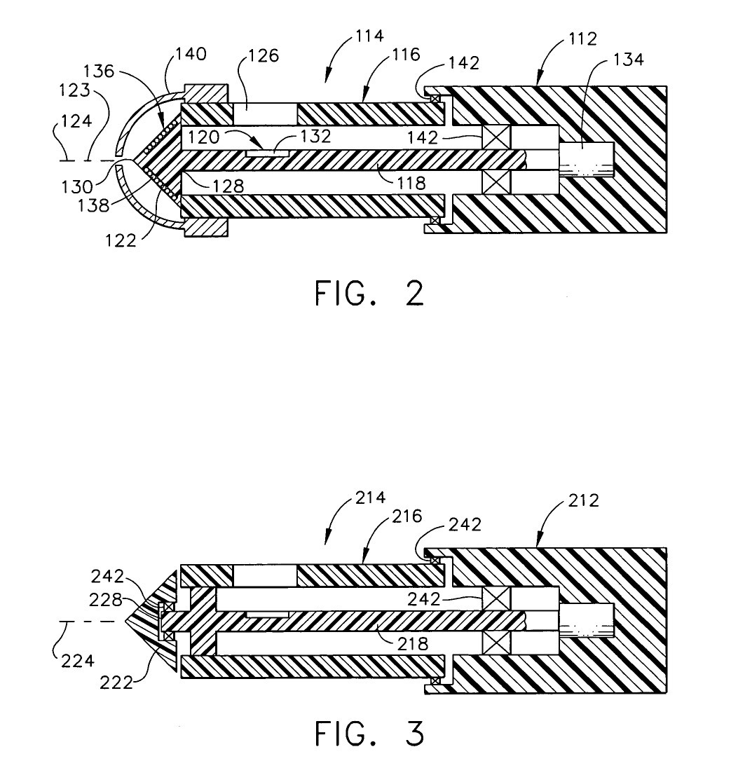

Referring now to the drawings, FIGS. 1-2 illustrate a first embodiment of the present invention. A first expression of the first embodiment of FIGS. 1-2 is for an ultrasound medical treatment system 110 including a handpiece 112 and including an end effector 114. The end effector 114 is operatively connected to the handpiece 112 and is insertable into a patient. The end effector 114 includes a shaft 118, a medical ultrasound transducer assembly 120, and a shaft head 122. The shaft 118 has a longitudinal axis 123, has a distal end 128, and is rotatable about the longitudinal axis 124 with respect to the handpiece 112. The transducer assembly 120 is non-rotatably attached to the shaft 118 and is adapted to emit medical ultrasound. The shaft head 122 is attached to the distal end 128 of the shaft 118 and has a piercing tip 130 which is insertable into patient tissue. The phrase “operatively connected” includes, without limitation, the end effector 114 being attached directly to the handpiece 112 and includes the end effector 114 being attached to an intervening tube (not shown) which is attached to the handpiece 112. The phrase “insertable into patient tissue” includes, without limitation, interstitially insertable into patient tissue. In one variation, the shaft head 122 and/or the piercing tip 130 are substantially coaxially-aligned with the longitudinal axis 123 and in another variation the shaft head and/or the piercing tip are not so aligned.

A second expression of the first embodiment of FIGS. 1-2 is for an ultrasound medical treatment system 110 including a handpiece 112 and including an end effector 114. The end effector 114 is operatively connected to the handpiece 112 and is insertable into a patient. The end effector 114 includes a sheath 116, a shaft 118, a medical ultrasound transducer assembly 120, and a shaft head 122. The sheath 116 has a longitudinal axis 124 and has an acoustic window 126. The shaft 118 is substantially coaxially-aligned with the longitudinal axis 124, is circumferentially surrounded by the sheath 116, has a distal end 128, and is rotatable about the longitudinal axis 124 with respect to the handpiece 112. The transducer assembly 120 is non-rotatably attached to the shaft 118, is adapted to emit medical ultrasound, and is disposed to emit the medical ultrasound through the acoustic window 126. The shaft head 122 is substantially coaxially-aligned with the longitudinal axis 124, is attached to the distal end 128 of the shaft 118, and has a piercing tip 130 which is insertable into patient tissue. In one variation, the piercing tip 130 is substantially coaxially-aligned with the longitudinal axis 124 and in another variation the piercing tip is not so aligned.

One example, without limitation, of an “acoustic window” is an opening in the sheath. Another example is an area of the sheath made from acoustically-transmissive materials, such materials being well known to those skilled in the art. In an additional example, the entire sheath is an acoustic window. In a further example, a thinner part of the sheath is an acoustic window. In yet another example, the sheath is shrunk over (and thereby reinforces) an acoustic window. Other examples are left to the artisan.

In one example of the second expression of the first embodiment of the invention of FIGS. 1-2, the sheath 116 (including its longitudinal axis 124) and the shaft 118 are flexible. In one variation, the end effector 114 is an articulated end effector. In one method, the end effector 114 is endoscopically, laparoscopically, or open-surgery inserted into the patient. Other avenues of patient insertion of the end effector 114 are left to those skilled in the art.

In one employment, the transducer assembly 120 includes an ultrasound transducer 132 adapted to medically image and/or medically treat patient tissue. An ultrasound transducer includes either a single ultrasound transducer element or an array of ultrasound transducer elements, as is known to those skilled in the art. In one construction, not shown, the shaft 118 is a tube containing wires leading to the ultrasound transducer elements. Examples of ultrasound medical imaging and medical treatment of patient tissue, include, without limitation, imaging of tumors, ablation of cancerous patient tissue, and hemostasis to stop abnormal bleeding and/or to stop blood flow to cancerous patient tissue.

In one enablement of the second expression of the first embodiment of FIGS. 1-2, the system 110 also includes a motor 134 operatively connected to the shaft 118 to rotate the shaft 118 about the longitudinal axis 124 with respect to the handpiece 112. In one construction, the motor 134 is disposed in the handpiece 112 as shown in FIG. 2. In another construction, not shown, the motor is disposed in the end effector. Other locations for the motor are left to the artisan. In a different enablement, the system does not include a motor.

In one arrangement of the second expression of the first embodiment of FIGS. 1-2, the system 110 also includes means 136 for ablating patient tissue in contact with the shaft head 122. In one construction, such means 136 includes a resistive heating element 138 which heats the shaft head 122. In another construction, such means 136 includes a heated fluid, such as heated water, which heats the shaft head 122. In a further construction, such means 136 includes an ablating chemical agent which is disposable on the outside of the shaft head 122. In an additional construction, such means 136 includes equipping the piercing tip 130 to emit monopolar and/or bipolar radio-frequency energy. Other such means 136 are left to the artisan.

In the same or a different arrangement, the system 110 also includes a protective cover 140 surrounding the shaft head 122. The protective cover 140 is self-opening exposing the shaft head 122 when the protective cover is pushed against patient tissue with a force greater than a threshold force. In one construction, the protective cover 140 is attached to the sheath 116. In one example, the protective cover 140 operates like a conventional protective cover of a conventional trocar, as can be understood by those skilled in the art.

In one illustration of the second expression of the first embodiment of FIGS. 1-2, the system 110 includes an ultrasound controller 144, wherein the ultrasound controller 144 is operatively connected to a foot-pedal power switch 146, as can be appreciated by the artisan. In one variation, the handpiece 112 includes a control knob 148 used to articulate the end effector 114 and includes a control button 150 used to activate the motor 134 to rotate the shaft 118 including the transducer assembly 120. In one configuration, not shown, water between the shaft 118 and the sheath 116 provides acoustic coupling between the transducer assembly 120 and the acoustic window 126. In one implementation, the handpiece 112 is translationally and rotationally fixed during any medical imaging/and/or treatment (such as by a user's hand or a mechanical arm assembly). In another implementation, the handpiece 112 is translated and/or rotated to compensate for any translational and/or rotational movement of the patient tissue (e.g., caused by respiration and/or heart beat) during any medical imaging and/or treatment.

Examples of acoustically-transmissive materials include, without limitation, PET [polyethylene terephthalate] (such as 0.001-inch-thick PET for a fully-circumferential acoustic window), Nylon 6, 11 or 12, TPX [methylpentene copolymer] and flouropolymers such as PTFE [polytetrafluoroethylene], FEP [fluorinated ethylene propylene], PFA [perfluoroalkoxy], PVDA [polyvinylidene acetate], ETFE [ethylene tetrofluoroethylene], polyurethane and polyethylene (high and low density). Shaft and sheath materials, for flexible shafts and sheaths, include, without limitation, Nitinol, polyimide, reinforced polyimide, Nylon, Pebax, silicone, reinforced silicone, polyurethane, polyethylene, flouropolymers and coiled metals (e.g., coiled stainless steel). When additional rotational stabilization of the sheath and/or the shaft head is desired, in one example, a surface modification (such as, without limitation, fixed ribs, fixed teeth, tapping features, grit blasting, rough machining, diamond-coating, acid etch, plasma-sprayed titanium, plasma-sprayed hydroxyupatite, microgrooves, porous coatings and rough coatings) is provided on all or part of the sheath and/or on all or part of the shaft head. In one assemblage, a shrink-tube attachment is used to join, for example, a polyimide non-acoustic-window portion of the sheath (or even the shaft head) to a fully-circumferential PET acoustic-window portion of the sheath.

In one arrangement of the second expression of the first embodiment of FIGS. 1-2, the sheath 116 and the shaft head 122 are rotatable about the longitudinal axis 124 with respect to the handpiece 112. In one variation, the sheath 116 is non-rotatably attached to the shaft 118 and rotatably attached to the handpiece 112, and the shaft head 122 is non-rotatably attached to the distal end 128 of the shaft 118. Rotatable couplings 142 are shown in FIG. 2 to indicate rotatable attachment of parts. Examples of a rotatable coupling, without limitation, are a ball-bearing coupling and a fluid seal (e.g., an O-ring and a plastic lathe-cut seal). Other examples are left to the artisan. Other variations, etc. to accomplish the desired rotatability and non-rotatability of parts are left to those skilled in the art. In an expansion of the second embodiment, the sheath 116 is omitted from the end effector 114.

A second embodiment of the end effector 214 is shown in FIG. 3. In this embodiment, the sheath 216 is rotatable about the longitudinal axis 224 with respect to the handpiece 212, and the shaft head 222 is non-rotatable with respect to the handpiece 212 when the shaft head 222 is non-rotatably fixed in patient tissue. In one arrangement, there is included an encoder (not shown) which relates the rotational position of the shaft to the patient tissue. In one variation, the sheath 216 is non-rotatably attached to the shaft 218 and rotatably attached to the handpiece 212, and the shaft head 222 is rotatably attached to the distal end 228 of the shaft 218. Rotatable couplings 242 are shown in FIG. 3 to indicate rotatable attachment of parts. Other variations, etc. to accomplish the desired rotatability and non-rotatability of parts are left to those skilled in the art. In an expansion of the second embodiment, the sheath 216 is omitted from the end effector 214.

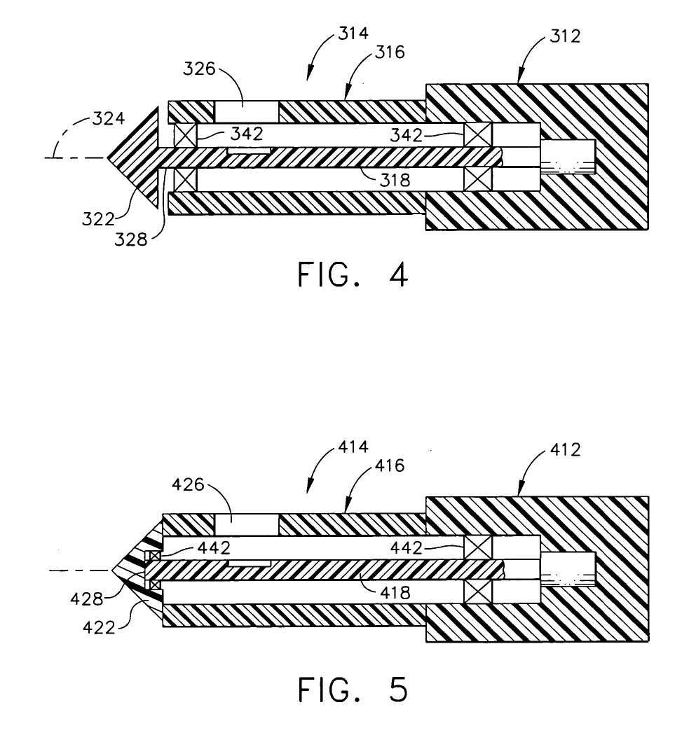

A third embodiment of the end effector 314 is shown in FIG. 4. In this embodiment, sheath 316 is non-rotatable with respect to the handpiece 312, and the shaft head 322 is rotatable about the longitudinal axis 324 with respect to the handpiece 312. In one variation, the shaft head 322 is non-rotatably attached to the shaft 318, and the shaft 318 is rotatably attached to the handpiece 312. In one design, the acoustic window 326 is a fully-circumferential acoustic window. In one modification, the shaft 318 proximal the distal end 328 is rotatably attached to the sheath 316. Rotatable couplings 342 are shown in FIG. 4 to indicate rotatable attachment of parts. Other variations, modifications, etc. to accomplish the desired rotatability and non-rotatability of parts are left to those skilled in the art.

A fourth embodiment of the end effector 414 is shown in FIG. 5. In this embodiment, the sheath 416 and the shaft head 422 are non-rotatable with respect to the handpiece 412. In one modification, not shown, the shaft head 422 is non-rotatably attached to the acoustic window 426 of the sheath 416. In one variation, the shaft head 422 is non-rotatably attached to the sheath 416 and rotatably attached to the distal end 428 of the shaft 418, and the sheath 416 is non-rotatably attached to the handpiece 412. In one design, the acoustic window 426 is a fully-circumferential acoustic window. In one modification, the shaft 418 is rotatably attached to the handpiece 412. Rotatable couplings 442 are shown in FIG. 5 to indicate rotatable attachment of parts. Other variations, modifications, etc. to accomplish the desired rotatability and non-rotatability of parts are left to those skilled in the art.

A fifth embodiment of the end effector 514 is shown in FIG. 5. In this embodiment, the ultrasound medical system also includes a suction sleeve 552. The suction sleeve 552 is non-rotatably attached to the handpiece 512 and circumferentially surrounds the sheath 516. The sheath 516 is non-rotatably attached to the shaft head 522 and is rotatably attached to the suction sleeve 552. The shaft head 522 is non-rotatably attached to the distal end of the shaft 518. In one variation, the suction sleeve 552 has a distal end, the acoustic window 526 has a proximal end, and the distal end of the suction sleeve 552 is circumferentially-suroundingly disposed at, or proximal to, the proximal end of the acoustic window 526. In an alternate embodiment, not shown, a suction sleeve is added to the embodiments of FIGS. 2 through 5.

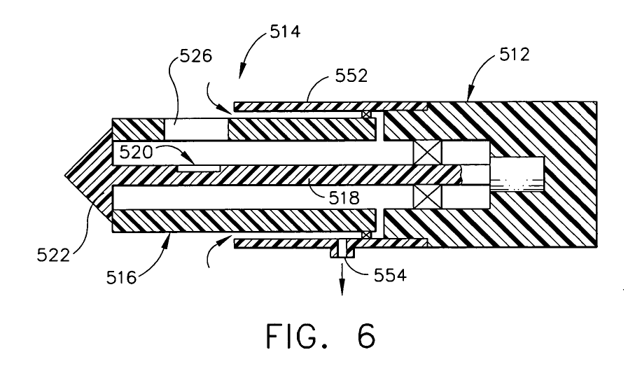

In one application of the fifth embodiment of FIG. 5, suction at the distal end of the suction sleeve 552 between the suction sleeve 552 and the sheath 516 provides longitudinal immobilization of the sheath 516 (and rotational stabilization in examples where the sheath does not rotate) which helps in precisely positioning the ultrasound source (i.e., the medical ultrasound transducer assembly 520) for ultrasound imaging and/or treatment. In one arrangement, the suction sleeve 552 includes a port 554, wherein the suction action is indicated by the unnumbered arrows in FIG. 6. The suction sleeve 552 is one example of means for additionally immobilizing the sheath 516 when the sheath 516 is inserted into patient tissue. Other such means, not shown, include the previously-described sheath surface modifications, suction holes in the sheath, and deployable needle-like or soft anchors. A further such means includes the sheath being a balloon sheath (including a weeping balloon sheath) adapted to expand against surrounding patient tissue for better stabilization and acoustic coupling. An additional such means is a separate inflatable balloon (including a weeping balloon). In one variation, the weeping balloon is used to deliver drug(s) and/or chemical adjuvants to the treatment employed, including drugs activated by ultrasound (e.g., by destruction of drug-containing liposomes) delivered from the medical ultrasound transducer assembly.

It is noted that examples, arrangements, enablements, etc. of the second expression of the first embodiment (such as the addition of a motor) are equally applicable to one or more or all of the second through fifth embodiments.

Several benefits and advantages are obtained from one or more of the embodiments of the ultrasound medical system of the invention. The rotatable shaft having the attached medical ultrasound transducer assembly provides a rotatable ultrasound source for medically imaging and/or medically treating a greater volume of patient tissue than those systems having non-rotatable ultrasound sources. The shaft head having the piercing tip provides longitudinal immobilization (and rotational stabilization in examples where the shaft head does not rotate) of the ultrasound source (i.e., the medical ultrasound transducer assembly) to patient tissue for precise positioning for ultrasound imaging and/or treatment. Use of an optional self-opening protective cover protects patient tissue from unintended contact with the piercing tip before and after ultrasound imaging and/or treatment as the ultrasound source is guided within the patient to and from the treatment site. Optional ablation of patient tissue in contact with the shaft head, along the end effector insertion track, sterilizes such patient tissue and is useful, for example, when the shaft head passes through cancerous tissue which is to be medically treated with the ultrasound source.

While the present invention has been illustrated by a description of several embodiments, it is not the intention of the applicant to restrict or limit the spirit and scope of the appended claims to such detail. Numerous other variations, changes, and substitutions will occur to those skilled in the art without departing from the scope of the invention. For instance, the ultrasound medical system of the invention has application in robotic assisted surgery taking into account the obvious modifications of such systems, components and methods to be compatible with such a robotic system. It will be understood that the foregoing description is provided by way of example, and that other modifications may occur to those skilled in the art without departing from the scope and spirit of the appended claims.

1. An ultrasound medical system comprising:
a) a handpiece; and
b) an end effector operatively connected to the handpiece, insertable into a patient, and including:
(1) a shaft having a longitudinal axis, having a distal end, and rotatable about the longitudinal axis with respect to the handpiece;
(2) a medical ultrasound transducer assembly non-rotatably attached to the shaft and adapted to emit medical ultrasound; and
(3) a shaft head attached to the distal end of the shaft and having a piercing tip which is insertable into patient tissue.

2. An ultrasound medical system comprising:
a) a handpiece; and
b) an end effector operatively connected to the handpiece, insertable into a patient, and including:
(1) a sheath having a longitudinal axis and having an acoustic window;
(2) a shaft substantially coaxially-aligned with the longitudinal axis, circumferentially surrounded by the sheath, having a distal end, and rotatable about the longitudinal axis with respect to the handpiece;
(3) a medical ultrasound transducer assembly non-rotatably attached to the shaft, adapted to emit medical ultrasound, and disposed to emit the medical ultrasound through the acoustic window; and
(4) a shaft head substantially coaxially-aligned with the longitudinal axis, attached to the distal end of the shaft, and having a piercing tip which is insertable into patient tissue.

3. The ultrasound medical system of claim 2, also including a motor operatively connected to the shaft to rotate the shaft about the longitudinal axis with respect to the handpiece.

4. The ultrasound medical system of claim 2, wherein the sheath and the shaft head are non-rotatable with respect to the handpiece.

5. The ultrasound medical system of claim 4, wherein the shaft head is non-rotatably attached to the sheath and rotatably attached to distal end of the shaft, and wherein the sheath is non-rotatably attached to the handpiece.

6. The ultrasound medical system of claim 5, wherein the acoustic window is a fully-circumferential acoustic window.

7. The ultrasound medical system of claim 5, also including a motor operatively connected to the shaft to rotate the shaft about the longitudinal axis with respect to the handpiece.

8. The ultrasound medical system of claim 5, wherein the shaft is rotatably attached to the handpiece.

9. The ultrasound medical system of claim 2, wherein the sheath is non-rotatable with respect to the handpiece, and wherein the shaft head is rotatable about the longitudinal axis with respect to the handpiece.

10. The ultrasound medical system of claim 9, wherein the shaft head is non-rotatably attached to the shaft, and wherein the shaft is rotatably attached to the handpiece.

11. The ultrasound medical system of claim 10, wherein the acoustic window is a fully-circumferential acoustic window.

12. The ultrasound medical system of claim 11, wherein the shaft proximal the distal end is rotatably attached to the sheath.

13. The ultrasound medical system of claim 10, also including a motor operatively connected to the shaft to rotate the shaft about the longitudinal axis with respect to the handpiece.

14. The ultrasound medical system of claim 2, wherein the sheath is rotatable about the longitudinal axis with respect to the handpiece, and wherein the shaft head is non-rotatable with respect to the handpiece when the shaft head is non-rotatably fixed in patient tissue.

15. The ultrasound medical system of claim 14, wherein the sheath is non-rotatably attached to the shaft and rotatably attached to the handpiece, and wherein the shaft head is rotatably attached to the distal end of the shaft.

16. The ultrasound medical system of claim 2, wherein the sheath and the shaft head are rotatable about the longitudinal axis with respect to the handpiece.

17. The ultrasound medical system of claim 16, wherein the sheath is non-rotatably attached to the shaft head and rotatably attached to the handpiece, and wherein the shaft head is non-rotatably attached to the distal end of the shaft.

18. The ultrasound medical system of claim 16, also including a suction sleeve non-rotatably attached to the handpiece and circumferentially surrounding the sheath, wherein the sheath is non-rotatably attached to the shaft head and rotatably attached to the suction sleeve, and wherein the shaft head is non-rotatably attached to the distal end of the shaft.

19. The ultrasound medical system of claim 18, wherein the suction sleeve has a distal end, wherein the acoustic window has a proximal end, and wherein the distal end of the suction sleeve is circumferentially-suroundingly disposed at, or proximal to, the proximal end of the acoustic window.

20. The ultrasound medical system of claim 2, also including means for ablating patient tissue in contact with the shaft head.

21. The ultrasound medical system of claim 2, also including a protective cover surrounding the piercing tip, attached to the sheath, and self-opening exposing the piercing tip when the protective cover is pushed against patient tissue with a force greater than a threshold force.

22. The ultrasound medical system of claim 2, wherein the sheath and the shaft are flexible.

23. The ultrasound medical system of claim 2, wherein the medical ultrasound transducer assembly includes an ultrasound transducer adapted to medically image and/or medically treat patient tissue.

24. An ultrasound medical system comprising:
a) a handpiece; and
b) an end effector operatively connected to the handpiece, insertable into a patient, and including:
(1) a sheath having a longitudinal axis and having an acoustic window;
(2) a shaft substantially coaxially-aligned with the longitudinal axis, circumferentially surrounded by the sheath, having a distal end, and rotatable about the longitudinal axis with respect to the handpiece;
(3) a medical ultrasound transducer assembly non-rotatably attached to the shaft, adapted to emit medical ultrasound, and disposed to emit the medical ultrasound through the acoustic window;
(4) a shaft head substantially coaxially-aligned with the longitudinal axis, attached to the distal end of the shaft, and having a piercing tip which is insertable into patient tissue; and
(5) a protective cover surrounding the piercing tip, attached to the sheath, and self-opening exposing the piercing tip when the protective cover is pushed against patient tissue with a force greater than a threshold force; and
c) a motor operatively connected to the shaft to rotate the shaft about the longitudinal axis with respect to the handpiece, wherein the motor is disposed in the handpiece.

25. An ultrasound medical system comprising:
a) a handpiece; and
b) an end effector operatively connected to the handpiece, insertable into a patient, and including:
(1) a sheath having a longitudinal axis and having an acoustic window;
(2) a shaft substantially coaxially-aligned with the longitudinal axis, circumferentially surrounded by the sheath, having a distal end, and rotatable about the longitudinal axis with respect to the handpiece;
(3) a medical ultrasound transducer assembly non-rotatably attached to the shaft, adapted to emit medical ultrasound, and disposed to emit the medical ultrasound through the acoustic window;
(4) a shaft head substantially coaxially-aligned with the longitudinal axis, attached to the distal end of the shaft, and having a piercing tip which is insertable into patient tissue; and
(5) a protective cover surrounding the piercing tip, attached to the sheath, and self-opening exposing the piercing tip when the protective cover is pushed against patient tissue with a force greater than a threshold force;
c) a motor operatively connected to the shaft to rotate the shaft about the longitudinal axis with respect to the handpiece, wherein the motor is disposed in the handpiece; and
d) means for ablating patient tissue in contact with the shaft head.
